(19) United States
(12) Patent Application Publication
(10) Pub. No.: US 2009/0099655 A1
(43) Pub. Date: Apr. 16, 2009

(54) POLYMERIC HYDROGEL

(76) Inventor: Michael Kircheisen, Hartheim (DE)

Correspondence Address: RANKIN, HILL & CLARK LLP, 38210 Glenn Avenue, WILLOUGHBY, OH, 44094-7808, US

(21) Appl. No.: 11/908,473

(22) PCT Filed: Mar. 16, 2005
(86) PCT No.: PCT/IB05/00691

§ 371 Date: Oct. 17, 2007

(51) Int. Cl. C08F 226/10; A61F 2/12; A61B 17/00

(52) U.S. Cl. 623/8; 526/263; 606/213

(57) ABSTRACT

The invention relates to a hydrogel polymer produced by polymerising an acrylic acid ester with an N vinylpyrrolidene and wherein less than 100% copolymer ester groups are saponified in such a way that free carboxyl groups are formed. The hydrogel polymer makes it possible to produce reliable long-lasting implants which are used, in particular, for increasing breasts or filling cutaneous wrinkles, are adaptable to each application and have a sufficiently high swelling coefficient.

The following invention relates to a polymeric hydrogel of the introductory portion of claim 1 and to its use in accordance with claim 10.

The inventive polymeric hydrogels have proven their value especially as permanent implant for enlarging the breast or for filling up large skin folds. The function of the permanent implant consists therein that it is inserted in a state of relative dryness at the desired place in the body and then, by taking up bodily fluids, swells up to a specified size, which is determined by the coefficient of swelling of the material

Some other materials have already become known for this purpose from the prior art. However, they have the disadvantage of being absorbed in the body. Other known implants, such as so-called tissue expanders, are used only temporarily, so that a permanent effect cannot be achieved with them.

It is an object of the invention to overcome the above-mentioned disadvantages of the prior art and to create a safe, permanent implant, which can be adapted to the respective application and has a sufficiently high coefficient of swelling.

Pursuant to the invention, this is accomplished with a polymeric hydrogel having the distinguishing features of claim 1.

In comparison to the prior art, permanent implants, which contain the inventive, polymeric hydrogel, have, for instance, the following advantages:
high biocompatibility/do not break down;
due to a water uptake of up to 98.5%, a very small amount of foreign material is introduced;
shortened surgery times;
simplified surgical method and, with that, a reduced risk of infection;
smaller incisions and, with that, a reduction in the surgery-related scar formation;
may be injected or introduced as a solid material;
surgery possible under a local anesthetic
no silicone or other coating required and
can be metered out in small steps and a further implant can be introduced later on.

Especially methacrylate esters, preferably methyl methacrylates, have proven their value for producing the polymeric hydrogel. Advantageously, more than 35% and preferably more than 40% of the ester groups of the polymeric hydrogel are saponified. Advantageously, less than 75% and, preferably, less than 70% of the ester groups are saponified. In this connection, the degree of saponification of the hydrogel may be determined by known analytical methods.

In the case of a special embodiment, the ratio by weight of acrylate ester to N-vinylpyrrolidone ranges from 0.1 to 0.67 and preferably from 0.33 to 0.53.

For swelling in a physiological salt solution, the coefficient of swelling of the polymeric hydrogel should advantageously be 20 to 80 and preferably 35 to 60.

For the applications under consideration, the preferred ranges given for the degree of saponification, the ratio by weight of the two components and the swelling coefficients have led to permanent implants, which behave optimally for the applications under consideration. The coefficient of swelling can be varied and, with that, the desired softness of the permanent implant adjusted by adjusting the degree of saponification. At a high degree of saponification, the hydrogels obtained are very soft and, with that, comparable to fatty tissue. If a stiffer implant is desired, a lesser degree of saponification should be selected.

For a special embodiment, the polymeric hydrogel has a pH ranging from 6.4 to 7.9 and preferably from 6.7 to 7.3. It was possible to achieve optimum tissue compatibility in this pH range.

For a further embodiment, the residual monomer content of the polymeric hydrogel is less than 0.01% by weight and preferably less than 0.001% by weight. These reduced amounts of unreacted residual monomers are obtained by washing the hydrogel repeatedly with water and, in conjunction with the small amount of hydrogel to be implanted, have proven to be biocompatible to a high degree, so that the danger of allergic reactions and other rejection reactions of the body is minimized.

For a further embodiment, the copolymer has the property of not being degraded or absorbed in an aqueous medium. The advantage of this embodiment consists therein that harmful degradation products are not formed in the body.

The inventive, polymeric hydrogel advantageously finds use as a swellable permanent implant to increase the breast or to fill up folds in the skin. Before it swells by absorbing bodily fluids, the permanent implant advantageously has a spherical configuration and the diameter of the spheres advantageously is 6 to 15 mm. Typically, spheres with a diameter of 8 to 12 mm are used. The spherically shaped permanent implant is particularly suitable for breast augmentation. Due to the spherical shape, an advantageous connection of the individual spheres in the swollen state is ensured in the case of several partial implants.

In the case of an alternative embodiment, a permanent implant has a cylindrical configuration before it swells by taking up bodily fluid and the diameter of the circular cylinder preferably is 0.5 to 3 mm. Typically, cylinders with a circular diameter of 1.5 to 2.5 mm are used. The ratio of the length L to the diameter D of the cylinder advantageously is between 1.5 and 5.0 and preferably between 2.0 and 4.0. Cylindrical implants of this configuration have led to particularly good results when skin folds are filled up.

For a further embodiment, the permanent implant advantageously consists of 2 to 30 and preferably of 3 to 20 partial implants. Before swelling by absorbing water, the permanent implant or the individual partial implants typically have a volume of 0.01 to 2.00 mL. Moreover, before it swells by absorbing bodily fluids, the permanent implant advantageously has a water content of less than 15% by weight. A preferred range for the water content is 5 to 10% by weight.

For a further embodiment, the ratio of the percentage P of saponified ester groups to the average diameter D of the permanent implant or of the individual partial implant ranges from 2.4 to 12.0 and preferably from 3.0 to 10.0.

For a further embodiment, the permanent implant or the individual partial implants have no coating. Compared to known implants with a coating, especially a silicone coating, the following advantages arise:
no additional foreign material is brought into the body, so that interactions with such foreign material are precluded;
less capsule formation and
no solid whole part, that is, individual dosing becomes possible and, with that, scar formation is minimized.

In the following, some examples for producing the inventive hydrogel and for clinical application cases of the permanent implants, produced therewith, are described in greater detail by means of surgery techniques involving them:

### EXAMPLE 1

Methyl methacrylate and N-vinylpyrrolidone were polymerized in a ratio by weight of 1:0.43 to semi-finished products, from which spheres with a diameter of 10 mm, corresponding to a volume of approximately 0.5 mL, were produced. After the residual monomers and oligomers were washed out with water, the spheres are converted by saponification with sodium hydroxide solution into an ionic hydrogel.

At the end of the production process, a solid starting sphere with a volume of about 0.5 mL, a residual moisture content (pyrogen-free water) of approximately 13% by weight, a residual methyl methacrylate monomer content of 0.00022% by weight and a residual N-vinylpyrrolidone monomer content of 0.00014% by weight was obtained.

For the production, the saponification time was selected so that a degree of saponification of approximately 50% and a coefficient of swelling of approximately 40 were achieved, that is, so that the volume of a sphere, swollen in a physiological salt solution, was 40 times that of the unswollen starting sphere.

The spherical implants were used in accordance with the description of the operation below for breast augmentation under local anesthesia:
a) To begin with, the mammary gland outline is indicated with the patient standing.
b) An IV is started.
c) Dormicum sedation applied.
d) Cefuroxim (1.5 g) administered as a single shot antibiotic.
e) After that, infiltration of the planned incision in the inframammary fold as well as of the breast base with local anesthetic.
f) Now the skin is severed to a length of 2 cm in the inframammary fold.
g) From there, preparation of the implant pocket under visual control with the electrocauterizer (epipectoral, if the gland is of sufficient size, subpectoral in the case of a thin soft tissue mantle). Careful hemostasis with the bipolar forceps.
h) After that, rinsing the pocket with salt solution.
i) A number of individual spherical implants of the inventive polymeric hydrogel are now brought in to the finished pocket, the number corresponding to the final volume established pre-operatively with the patient.
j) Subsequently, the implant pocket is closed with 3x0 Vicryl for the adaptation of gland and fascia, 4x0 Vicryl subcutaneous and 4x0 Prolene for the intracutaneous skin suture.
k) Applying Tegaderm plaster.
l) Applying a body belt for compression for 24 hours.

In the present case, a final volume of 200 mL was agreed upon with the patient pre-operatively. In order to achieve this, 10 spheres with a starting volume of approximately 5 mL were used. The aimed for final volume was attained after about 10 days by the absorption of bodily fluid.

### EXAMPLE 2

Methyl methacrylate and N-vinylpyrrolidone were polymerized in a ratio by weight of 1:0.25 to semi-finished products, from which cylindrical parts with a diameter of the circular cylinder of 2 mm and a length of the cylinder of 8 mm, corresponding to a volume of approximately 0.025 mL, were prepared. After residual monomers and oligomers were flushed out with water, the cylinders were converted by saponification with sodium hydroxide solution into an ionic hydrogel.

At the end of the manufacturing process, a solid starting cylinder, with a volume of 0.025 mL and a residual moisture content (pyrogen-free water) of approximately 10% by weight was obtained once again.

For the preparation, the saponification time was selected so that a degree of saponification of approximately 35% and a coefficient of swelling of approximately 20 were obtained, that is, that the volume of a cylinder, swollen in physiological salt solution, was 20 times that of the unswollen starting cylinder.

The cylindrical implants were used in accordance with the description of a surgery below for fold therapy (nasolabial fold, angle of the mouth fold), lip augmentation, filling up a blemish in the face area. The number of cylinders, inserted by means of an applicator cannula, was determined on the basis of the size of the defect and established pre-operatively.
a) To begin with, the planned stab incision (2 mm) was indicated at the lateral angle of the mouth (or other localization in the case of filling a defect).
b) Subsequently, infiltration of the planned incision with a little local anesthetic.
c) Now stab incision with a No. 11 blade.
d) Inserting the application cannula and advancing it subcutaneously into the target area (nasolabial fold, angle of the mouth fold, upper lip, lower lip).
e) Setting a number of pellets stepwise, commencing at the uppermost target point and continuing in the direction of the incision (the number corresponds to the previously established length).
f) At the end, closure of the skin incision with a 5x0 Prolene single button suture.

1. Polymeric hydrogel, obtained by copolymerization of an acrylate ester with N-vinylpyrrolidone, wherein
A) less than 100% of the ester groups of the copolymer are saponified to free carboxyl groups; and
B) the ratio by weight of acrylate ester to N-vinylpyrrolidone ranges from 0.1 to 0.67; and
C) when swelling said hydrogel in a physiological salt solution, the coefficient of swelling is 20 to 80.

2. The polymeric hydrogel of claim 1, wherein the acrylate ester is a methacrylate ester.

3. The polymeric hydrogel of claim 1, wherein more than 35% of the ester groups are saponified.

4. The polymeric hydrogel of claim 1, wherein less than 70% of the ester groups are saponified.

5. The polymeric hydrogel of claim 1, wherein the ratio by weight of acrylate ester to N-vinylpyrrolidone ranges from 0.33 to 0.53.

6. The polymeric hydrogel of claim 1, wherein for swelling in a physiological salt solution, the coefficient of swelling is 35 to 60.

7. The polymeric hydrogel of claim 1, wherein pH ranges from 6.7 to 7.3.

8. The polymeric hydrogel of claim 1, wherein it contains less than 0.001% by weight of a residual monomer.

9. The polymeric hydrogel of claim 1, wherein the copolymer is not degradable or absorbable in an aqueous medium.

10. Use of the polymeric hydrogel of one of claim 1 as a swellable permanent implant for enlarging the breast or for filling folds in the skin.

11. The use of claim 10, wherein the permanent implant, before it swells by taking upper bodily fluid, has a spherical configuration and the diameter of the sphere is 6 to 15 mm.

12. The use of claim 10, wherein the permanent implant, before it swells by taking up bodily fluid, has a cylindrical configuration and the diameter of the circular cylinder is 0.5 to 3 mm.

13. The use of claim 12, wherein the ratio of the length L of the cylinder to the diameter D of the circular cylinder ranges from 2.0 to 4.0.

14. The use of claim 10, wherein the permanent implant consists of 3 to 20 partial implants.

15. The use of one claim 10, wherein the permanent implant or the individual partial implants, before they swell by taking up water, have a volume of 0.01 to 2.00 mL

16. The use of claim 10, wherein the permanent implant, before it swells by taking up bodily fluid, has a water content of less than 15% by weight.

17. The use of claim 10, wherein the ratio of the percentage P of saponified ester groups to the average diameter D of the permanent implant or the individual partial implants ranges from 3.0 to 10.0.

18. The use of claim 10, wherein permanent implants or the individual partial implants do not have a coating.

19. The polymeric hydrogel of claim 1, wherein the acrylate ester is methyl methacrylate.

20. The polymeric hydrogel of claim 1, wherein more than 35% and preferably more than 40% of the ester groups are saponified.
